(19) United States
(12) Patent Application Publication
(10) Pub. No.: US 2014/0323811 A1
(43) Pub. Date: Oct. 30, 2014

(54) METHODS AND APPARATUS FOR RETRACTING TISSUE

(71) Applicant: Invuity, Inc., San Francisco, CA (US)

(72) Inventors: Stephen DeSantis, Laguna Niguel, CA (US); Alex Vayser, Mission Viejo, CA (US); Douglas Rimer, Los Altos Hills, CA (US); Ryan Boucher, San Francisco, CA (US); David Wayne, Watsonville, CA (US)

(73) Assignee: Invuity, Inc., San Francisco, CA (US)

(21) Appl. No.: 14/264,406

(22) Filed: Apr. 29, 2014

Related U.S. Application Data
(60) Provisional application No. 61/817,508, filed on Apr. 30, 2013.

(51) Int. Cl. A61B 17/02; A61B 1/06

(52) U.S. Cl. CPC A61B 17/0218; A61B 1/06; A61B 2017/0042
USPC 600/213; 600/245

(57) ABSTRACT

A surgical retractor includes a retractor blade and a gripping element releasably coupled to the retractor blade. The gripping element has a textured surface for engaging and gripping tissue. The gripping element may be released from the retractor blade intraoperatively. The surgical retractor may also have an optical illumination element such as an optical waveguide that is magnetically and releasably coupled to the retractor.

### CROSS-REFERENCE

The present application is a non-provisional of, and claims the benefit of U.S. Provisional Patent Application No. 61/817,508 (Attorney Docket No. 40556-729.101) filed Apr. 30, 2013; the entire contents of which are incorporated herein by reference.

### BACKGROUND OF THE INVENTION

1. Field of the Invention

The present invention generally relates to medical instruments and methods of use. More specifically, the present invention relates to surgical retractors used during surgery or other medical procedures for retracting or otherwise manipulating tissue as well as illuminating the tissue.

During the course of many open surgical procedures retractors are used to pull or manipulate tissue such as muscle, fat, and even delicate anatomy like vasculature or nerves. A retractor typically has a handle for grasping and manipulation by the surgeon and a long blade for engaging the tissue. The tissue is often pulled or pushed apart to create working access to the surgical field and develop the necessary exposure. Many of these surgical retractors or surgical instruments are machined from stainless steel, aluminum or even titanium.

One of the challenges with many of these instruments, especially retractors having long blades, is that the tip of the blade may slip due to low friction between the polished metal surface of the retractor blade and the tissue. Currently available commercial instruments attempt to address this challenge by adding various teeth or surface features to the distal end of the blade in order to create a better grip with tissue. In certain situations this approach works, however, as the surgeon works through various layers of tissue, different teeth or surface features may be required to grasp different tissue. For example, coarser teeth may be required to grasp fatty tissue, versus finer teeth for muscle or other more delicate tissue such as nerves or blood vessels. Thus, if the surgeon wants to modify the grip by increasing or decreasing the length or texture of the teeth based on the anatomy, the surgeon must look for another retractor, which may not exist. Hospitals must therefore maintain a large inventory of different retractors. Thus, there is a need to provide the surgeon devices having different textures for retraction of different tissues. Additionally, there is a need to provide the surgeon devices having different amounts and patterns of texture that also accommodate various tissues as well as different retractor blade configuration. At least some of these challenges will be addressed by the present disclosure.

In addition to gripping tissue during retraction, many surgical procedures require illumination. Current commercial devices may use fiber optic cables coupled to a surgical retractor to illuminate the surgical field. However, these instruments do not always deliver light efficiently to the target, can generate excessive amounts of heat, and often require constant repositioning to deliver the light to the desired location. Other commercial devices may include optical waveguides which snap or otherwise are coupled to a surgical retractor which has predesigned features for attachment of the illumination component. Often, the optical waveguide is either fixed to the retractor blade or it may not be easy or convenient to move the waveguide position relative to the retractor blade. Thus, it would be desirable to provide a surgical retractor that has a lighting component that is easily attached and detached from the surgical retractor blade, and that has the ability to be easily repositioned. For example, it would be desirable if the lighting component could be moved proximally and distally along the retractor blade in order to adjust its position and delivery of the light therefrom. It would also be desirable if the lighting component could be adjusted in other directions as well, including but not limited to laterally or medially along the blade width, or rotating the lighting component relative to the blade, or angling it. Repositioning the optical component may also be useful in preventing damage to the optical component by moving it out of the way of other surgical instruments. One approach to addressing this challenge is to use an adhesive tape to adjustably couple the illumination component with the retractor blade. Other mechanical features may also be used for coupling, such as with screws, snaps, press fits, etc. However, these may be difficult and cumbersome to reposition. Therefore, it would be desirable to provide devices and methods that allow a user to easily attach, detach, and reposition the lighting component to the surgical retractor. At least some of these objectives will be satisfied by the present disclosure.

2. Description of the Background Art

Patents and publications related to tissue grasping features include but are not limited to US Patent Publication No. 2013/0035555; and U.S. Pat. Nos. 8,360,972; 7,909,761; 7,481,766; 7,017,581; 6,994,669; 6,419,175; and 5,709,646.

Patents and publications related to repositionable illumination components include but are not limited to US Patent Publication Nos. 2008/0058835; and 2007/0060795; and U.S. Pat. Nos. 8,343,048; 8,132,949; 7,306,559; and 6,113,536.

### SUMMARY OF THE INVENTION

The present invention generally relates to medical instruments and methods of use. More specifically, the present invention relates to surgical retractors used during surgery or other medical procedures for holding or otherwise manipulating tissue as well as illuminating the tissue.

In a first aspect of the present invention, a surgical retractor for retracting tissue comprises a retractor blade having a proximal end, a distal end, and a gripping element. The gripping element is releasably coupled to the retractor blade and has a textured surface adapted to engage and grip the tissue. The gripping element is adapted to be released from the retractor blade before, during or after surgery.

The gripping element may be adhesively coupled with the retractor blade. The retractor blade may have a rear surface for engaging the tissue and a front surface opposite thereto, and the gripping element may be disposed on the rear surface. The gripping element may be flexible and be configured to conform to the surface of the retractor blade. The gripping element may be continuously disposed on the retractor blade, or the gripping element may comprise a plurality of gripping pads disposed in a plurality of discrete regions on the retractor blade. The gripping element may extend substantially all the way between the proximal and distal ends of the retractor blade, or at least a portion of the retractor blade may remain uncovered by the gripping element.

The textured surface may be uniformly textured along the gripping element, or the textured surface may comprise a first textured region and a second textured region. The first textured region may be textured differently than the second textured region. The first textured region may be closer to the proximal end of the retractor blade than the second texture region, and the second textured region may be closer to the distal end than the first textured region. The first textured region may have a finer texture than the second textured region, and the second textured region may have a coarser texture than the first textured region. The textured surface may be configured to engage and grasp fat, muscle, blood vessels, nerves, or skin. The textured surface may comprise a plurality of gripping elements protruding outward from the retractor blade. The plurality of gripping elements may extend outward at an angle relative to an outer surface of the retractor blade. The angle may be perpendicular or orthogonal. The plurality of gripping elements may be arranged into a plurality of rows disposed on the retractor blade, or into a symmetric pattern. The gripping elements also may have different sizes such as heights and widths and they may be asymmetrically positioned along the retractor blade. The gripping element may be flexible and configured to conform to the retractor blade when disposed thereon. The gripping elements may comprise holes in a substrate coupled to the retractor blade, and the holes may be configured to receive tissue.

The retractor blade may have a recessed region for receiving the gripping element such that the gripping element is substantially flush with the retractor blade. The retractor blade may further comprise an optical waveguide coupled to the retractor blade, and the waveguide may be configured to deliver light toward the tissue. The retractor may also comprise a handle coupled to the proximal end of the retractor blade. Any of the embodiments may include a smoke evacuation element that is disposed adjacent a distal end of the retractor blade. The smoke evacuation element is preferably configured to remove fumes from a surgical field. Also, any of the embodiments may include an imaging element adjacent a distal end of the retractor blade. The imaging element is preferably configured to provide an image of a surgical field.

In another aspect of the present invention, a surgical method for retracting tissue comprises providing a surgical retractor blade and selecting a gripping element from a plurality of different gripping elements. The surgical retractor blade has a proximal end and distal end. The method also includes releasably coupling the selected gripping element with the surgical retractor blade and advancing the surgical retractor blade toward the tissue. The method also comprises engaging the gripping element with the tissue, and retracting the surgical retractor blade thereby retracting the tissue.

Releasably coupling may comprise adhesively coupling the selected gripping element with the surgical retractor blade. The method may further comprise uncoupling the selected gripping element from the surgical retractor blade and selecting a second gripping element from the plurality of gripping elements. The second gripping element may be releasably coupled with the surgical retractor blade. The gripping elements may also wrap around the retractor blade, either partially or completely, thus the gripping elements are not limited to being disposed on the back of the retractor blade.

Retracting the tissue may comprise retracting fat, muscle, nerves, blood vessels, or skin. The surgical retractor blade may have an optical illumination element such as an optical waveguide coupled thereto, and the method may further comprise illuminating the tissue with light from the optical illumination element. Releasably coupling the selected gripping element may comprise disposing the selected gripping element substantially all the way or partially between the proximal and distal ends of the surgical retractor blade. Releasably coupling the selected gripping element may comprise maintaining a portion of the surgical retractor blade uncovered by the selected gripping element.

The selected gripping element may have a first textured region and a second textured region. The first textured region may have a texture different than the second textured region, and releasably coupling the selected gripping element may comprise disposing the first textured region at a first end of the surgical retractor blade and disposing the second textured region at a second end of the surgical retractor blade opposite the first end. The first textured region may have a finer texture than the second textured region, and the second textured region may have a coarser texture than the first textured region. Releasably coupling may comprise disposing the first textured region adjacent the proximal end of the surgical retractor blade and disposing the second textured region adjacent the distal end of the surgical retractor blade. The gripping element may comprise a plurality of gripping pads, and releasably coupling may comprise affixing the plurality of gripping pads to the surgical retractor blade. Releasably coupling the selected gripping element with the surgical retractor blade may comprise conforming the selected gripping element to the surgical retractor blade. Releasably coupling may comprise disposing the selected gripping element in a recessed region of the surgical retractor blade so that the selected gripping element is substantially flush with the retractor blade. Any of the methods may include evacuating smoke from a surgical field with a smoke evacuation element that is disposed adjacent a distal end of the retractor blade. Any of the methods may also comprise imaging a surgical field with an imaging element that is disposed adjacent a distal end of the retractor blade.

In another aspect of the present invention, an illuminated surgical retractor for retracting tissue comprises a retractor blade having a proximal end and a distal end, an optical illumination element such as an optical waveguide configured to deliver light from the optical illumination element to the tissue, and a magnetic element coupled to the retractor blade and the optical illumination element so that the optical illumination element is releasably coupled to the retractor blade.

The magnetic element may comprise a plurality of magnets, and the magnetic element may be fixedly attached to one of the optical illumination element or the surgical retractor blade. The optical illumination element may be adjustably coupled with the surgical retractor blade such that the optical illumination element may be positioned between the proximal and distal ends of the surgical retractor blade. The illuminated surgical retractor may further comprise a magnetic or metallic adhesive strip disposed between the optical illumination element and the magnetic element, or between the surgical retractor blade and the magnetic element. A smoke evacuation element or an imaging element may be disposed adjacent a distal end of the retractor blade. The smoke evacuation element preferably removes fumes from a surgical field, and the imaging element preferably provides an image of a surgical field.

In still another aspect of the present invention, a surgical method for retracting tissue in a patient comprises providing a surgical retractor blade having a proximal end and a distal end and providing an an optical illumination element such as an optical waveguide. The method also includes magnetically coupling the optical illumination element with the retractor blade, advancing the retractor blade to the tissue, and retracting the tissue with the retractor blade. The tissue is then illuminated with light from the optical illumination element.

The method may further comprise adjusting position of the optical illumination element relative to the surgical retractor blade. Adjusting position may comprise uncoupling the optical illumination element from the surgical retractor blade, moving the optical illumination element relative to the surgical retractor blade, and re-coupling the optical illumination element magnetically with the surgical retractor blade. Adjusting position may also comprise moving the optical illumination element between the proximal and distal ends of the surgical retractor blade or laterally or medially therealong. The optical illumination element may also be rotated relative to the retractor blade. The magnetic coupling is advantageous because it is unaffected by water, blood, or other fluids encountered during surgery and thus holds the components together. This is unlike many adhesive strips which may fail if they get wet. Magnetically coupling may comprise coupling the optical illumination element with the surgical retractor blade using a plurality of magnets. The method may further comprise affixing a magnetic adhesive strip to the optical illumination element or the surgical retractor blade. In addition to magnets, electromagnets may also be used to couple the components together. An on-off switch allows the magnetic field to be turned on and off. Similarly, a controller may be used to control the strength of the magnetic field. The method may optionally include evacuating smoke or illuminating a surgical field with an illumination element, either of which may be disposed adjacent a distal end of the retractor blade.

In still another aspect of the present invention, a system for holding a surgical retractor comprises a clip comprising a first arm and a second arm opposite the first arm, the first arm having a first engagement element and the second arm having a second engagement element. The first and second arms are spaced apart by a distance that is sized to match a width of a retractor blade. The first engagement element is configured to engage a first edge of a retractor blade and the second engagement element is configured to engage a second edge of the retractor blade, thereby attaching the clip to the retractor blade. The system also includes a holding mechanism for holding an illumination element, the holding mechanism coupled to the clip.

The system may comprise the retractor blade or the illumination element. The illumination element may comprise a non-fiber optic optical waveguide. The system may also comprise a third arm and a fourth arm opposite the third arm. The third arm may have a third engagement element and the fourth arm may have a fourth engagement element. The third and fourth arms may be spaced apart by a distance that is sized to match the width of the retractor blade. The third engagement element may be configured to engage the first edge of the retractor blade and the fourth engagement element may be configured to engage the second edge of the retractor blade, thereby attaching the clip to the retractor blade.

The first arm and the second arm may be movable relative to one another thereby adjusting the distance. The first arm or second arm may be substantially linear or arcuate. The first arm may be rotatably engageable with the first edge or the second arm may be rotatably engageable with the second edge. The first engagement element or the second engagement element may comprise tabs. The tabs may be formed by folding over the arms over on themselves. The first edge of the retractor blade may be on an opposite side of the retractor blade as the second edge. T

The system may further comprise an arm coupleable with the clip. The arm may be configured to be coupled with an adjacent operating room table surface or other adjacent surface, and the arm may be configured to steadily hold the clip and retractor blade coupled thereto. The arm may be actuatable between a flexible movable configuration and a substantially inflexible immovable configuration for steadily holding the clip and retractor blade. The system may further comprise an imaging element adjacent a distal portion of the retractor blade for imaging a surgical field, or the system may comprise a smoke evacuation element for removing smoke or noxious fumes from a surgical site in which the retractor blade is disposed.

In yet another aspect of the present invention, a method for holding a surgical retractor blade comprises providing a surgical retractor blade having a first edge and a second edge opposite the first edge, providing a clip having a first arm and a second arm opposite the first arm, engaging the first arm with the first edge of the retractor blade, engaging the second arm with the second edge of the retractor blade, thereby holding the surgical retractor blade with the clip, and attaching a surgical instrument to the clip.

Engaging the first arm with the first edge may comprise engaging a tab on the first arm with the first edge, or engaging the second arm with the second edge may comprise engaging a tab on the second arm with the second edge. Attaching the surgical instrument may comprise attaching an illumination element to the clip. The method may further comprise illuminating a surgical field with light from the illumination element. The method may further comprise imaging a surgical field with an imaging element adjacent a distal end of the retractor blade. The method may further comprise evacuating smoke or fumes from a surgical field with a smoke evacuation element adjacent a distal end of the retractor blade. The method may further comprise coupling the clip to an arm that is coupled to an operating room table or adjacent surface. The arm preferably holds the clip and surgical instrument in a substantially fixed position. Engaging the first arm or second arm may comprise rotating the first arm into engagement with the first edge or rotating the second arm into engagement with the second edge.

These and other aspects and advantages of the invention are evident in the description which follows and in the accompanying drawings.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a side view of a surgical retractor.

FIGS. 2A-2B illustrate a a retractor having barbs or teeth.

FIG. 3A illustrates a perspective view of a retractor blade having textured features for grasping tissue.

FIG. 3B illustrates a side view of the embodiment in FIG. 3A.

FIG. 4 illustrates a side view of an exemplary embodiment of a retractor blade having textured features for grasping tissue.

FIG. 5 illustrates a rear view of an exemplary embodiment of a retractor blade having textured features for grasping tissue.

FIGS. 6A-6B illustrate side views of alternative embodiments of a retractor blade with textured features.

FIGS. 7A-7H illustrate various embodiments of gripping features.

FIGS. 8A-8B illustrate still other embodiments of gripping features.

FIGS. 9A-9B illustrate side views of alternative embodiments of magnetic coupling of an illuminator to a retractor blade.

FIGS. 10A-10B illustrate side views of alternative embodiments of magnetic coupling between an illuminator and a retractor blade.

FIGS. 11A-11C illustrate adjustment of an illuminator relative to a retractor blade.

FIGS. 12A-12B illustrate still other exemplary embodiments of magnetically coupling an illuminator to a retractor blade.

FIG. 13 illustrates an illuminated retractor magnetic coupling, grip features, smoke evacuation and an imaging element.

FIGS. 14A-14D illustrate an exemplary embodiment of a retractor blade clip.

FIG. 15 illustrates an optical waveguide coupled to a surgical retractor blade using a clip.

FIG. 16 is an exploded view of a clip.

FIGS. 17A-17D illustrate another exemplary embodiment of a clip.

FIG. 18 illustrates an optical waveguide coupled to a surgical retractor blade using a clip.

FIG. 19 illustrates an exploded view of the clip seen in FIGS. 17A-17D or FIG. 18.

FIG. 20 illustrates another exemplary embodiment of a clip for holding a retractor blade.

FIG. 21 illustrates an exemplary embodiment of an arm for holding a surgical instrument.

### DETAILED DESCRIPTION OF THE INVENTION

Tissue Gripping.

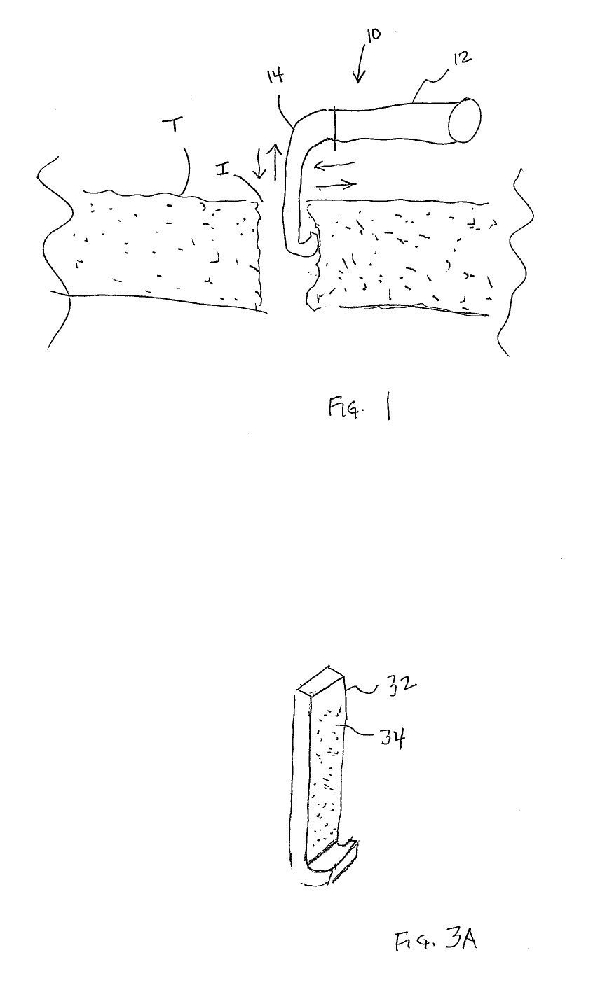

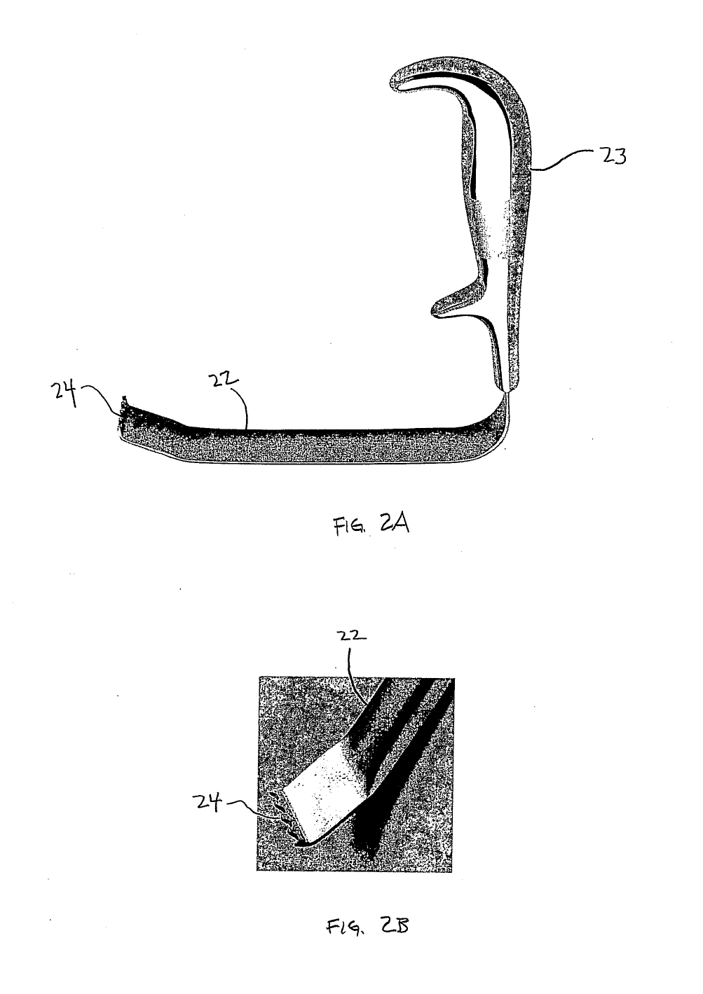

FIG. 1 illustrates a typical surgical retractor 10 having a handle 12 and retractor blade 14. The handle is often ergonomically designed to be held and grasped by a surgeon or other operator. The retractor blade 14 may have any number of configurations depending on the nature of the procedure being performed as well as the tissue being treated. The handle may be pushed, pulled, lifted upward, pushed downward, or otherwise manipulated in any desired direction, as indicated by the arrows, in order to engage the tissue T with the retractor blade 14 and to move the tissue in order to open the incision I and expose the surgical field. The retractor blade 14 may be fabricated from a polished metal and thus the surface can be slippery and this may make it difficult to grasp the tissue. Thus, barbs or teeth 24 or other textured surface features may be formed on the retractor blade 22 for more reliable tissue engagement as seen in FIG. 2A which shows a commercially available retractor having handle 23. FIG. 2B illustrates the barbs or teeth 24 more clearly. In other embodiments of retractor blades, the blade may have openings that allow tissue to pillow into the openings, thereby further ensuring that the retractor blade grips the tissue.

However, having one fixed pattern of barbs or textured features on a retractor blade does not always accommodate all tissues being treated. For example, the same length retractor blade may be used throughout a procedure and may not have to be changed, but it may be desirable to change the texturing on the blade during the same surgical procedure. In a surgical procedure, the same blade length may be used to retract muscle and fat. However, because fat is very slippery relative to muscle, the engaging surface of the retractor blade (typically the rear surface) may require a coarse texture to grasp the fat, while muscle is more delicate and thus the texturing may need to be finer. Additionally, as the procedure begins, the surgeon may use a retractor blade without texture for tissue that is closer to the skin surface. As the surgeon works down to create access to the surgical field, different layers of tissue are encountered and thus different grasping features may be required.

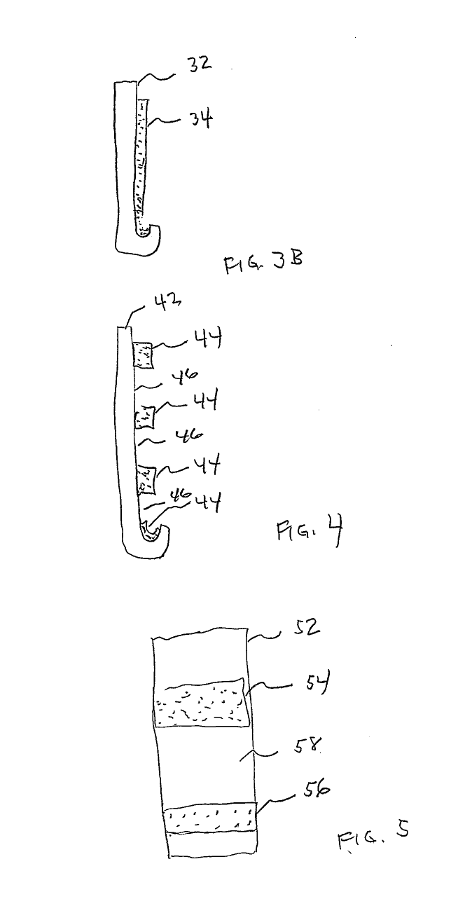

Thus, it would be desirable to provide a surgical retractor that has changeable texturing or grasping features on the retractor blade. FIG. 3A illustrates one exemplary embodiment of a retractor blade 32 having a removable and replaceable texturing 34 or grip strip on the rear surface of the retractor blade. In preferred embodiments, the texturing is easily applied and removed from the retractor blade. Thus, the texturing may be on a strip or substrate that can be adhesively applied to the retractor blade. Other attachment means may also be used such as by snap fitting, friction fitting, using fasteners, engagement with slots, etc. Thus, the texturing may be easily applied and removed from the retractor blade as needed. A variety of texturing ranging from fine to coarse may be supplied in a kit and the operator may select the desired coarseness and apply it to the retractor blade during one portion of a surgical procedure, and remove it and replace it with a different texture during another portion of the surgical procedure. The texturing may be formed from grit or other particles applied to the adhesively backed substrate, or various patterns and shapes may be formed in the substrate as will be discussed below. In the embodiment of FIG. 3A, the texturing is applied in a single strip along a majority of the retractor blade rear surface. The texturing may be uniform along the strip, or it may vary. FIG. 3B illustrates a side view of the strip 34 applied to the retractor blade 32. The strip 34 may be any thickness, but preferably is very thin so that it maintains a low profile of the retractor blade.

In some situations, rather than provide a continuous surface of texturing such as in FIGS. 3A-3B, it may be desirable to provide discrete regions of texturing on the retractor blade. FIG. 4 illustrates an exemplary embodiment of a retractor blade 42 having multiple regions 44 of texturing separated by untextured regions 46. The texturing may be the same in all regions, or the texturing may vary between regions. For example, the texturing toward the distal end of the retractor blade may be finer than the texturing in the proximal portions of the retractor blade which may be coarser. The discrete regions of texturing may be any shape or size.

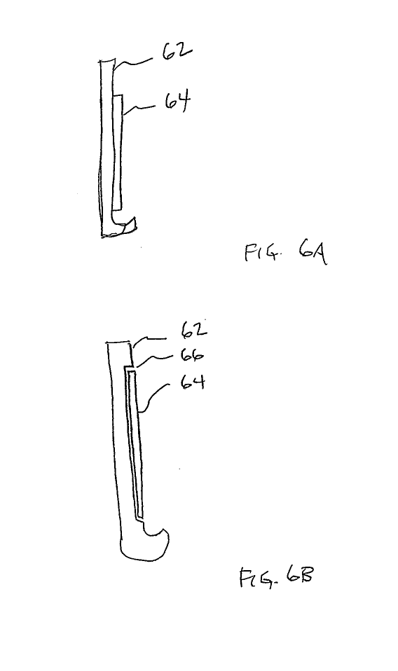

FIG. 5 illustrates another variation of texturing that may be used during a surgical procedure. In FIG. 5, the retractor blade 52 has a proximal region of texturing 54 and a distal region of texturing 56 separated by an untextured region 58. The proximal region of texturing 54 may be coarser than the distal region of texturing which may be finer. Thus, the retractor blade will have variable texturing along its length and the retractor blade will be able to grasp different types of tissue at different levels of the surgical incision. In this embodiment, texturing varies from coarse to finer the further distally the position on the retractor blade. For example, fat may be closer to the surface of the incision and muscle may be deeper, therefore the proximal textured region may grasp the fat, while the distal textured region may grasp the muscle. The textured regions preferably extend in bands all the way across the width of the retractor blade. However, in other embodiments, the bands may not extend all the way across the width or they may be in discrete regions across the width. In alternative embodiments, the texturing may be reversed so that texturing changes from coarse to finer as the position along the retractor blade moves proximally.

Profile is always an important factor for any surgical instrument. Too large of a profile will result in excessive space being occupied by the surgical instrument thereby making it challenging for the surgeon to insert his/her hands or other instruments into the surgical field. Therefore, it would be desirable to keep profile of the instrument minimized. FIGS. 6A-6B illustrate various options for reducing profile. For example, in FIG. 6A, the textured strip 64 is applied directly to the rear surface of the retractor blade 62. Thus, the textured strip will increase the profile of the device. However, if the strip is thin enough, this will not be significant. In FIG. 6B, a recessed region 66 is formed in the retractor blade 62 that accommodates some or all of the thickness of the textured strip 64. Thus, once the textured strip is applied to the retractor blade it will be flush with the rest of the retractor blade.

In some embodiments, the textured strip may be a sandpaper-like strip which is adhesively bonded to the retractor blade. Thus, random or symmetric patterns of grit are dispersed along the strip. In other embodiments, patterns of features may be formed into the strip which then can be adhesively bonded to the retractor blade or otherwise removably attached thereto.

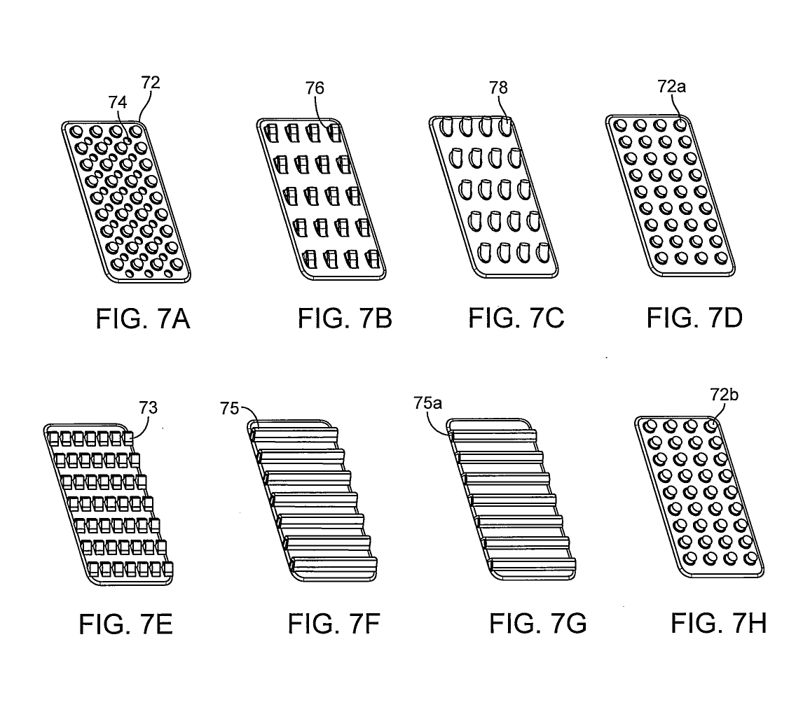

FIGS. 7A-7H illustrate alternative embodiments of texturing or surface features which may be formed into the strips to facilitate grasping of different tissues. For example, in FIG. 7A, alternative rows of cylindrical protuberances 72 and partial or through holes 74 are disposed on the substrate. The cylindrical protuberances grasp tissue by extending into the tissue, while the holes grasp tissue by allowing tissue to pillow up into the holes. FIG. 7B illustrates another embodiment where the surface features are rows of ramps 76 having an angled ramp surface and an angled face. FIG. 7C is similar to the embodiment of FIG. 7B except that the features are rows of angled ramps formed from cylindrical-like protuberances. FIG. 7D is similar to the previous embodiment in FIG. 7A and includes surface features formed from rows of cylindrical protuberances 72a. FIG. 7E illustrates surface features formed from rows of angled ramps 73. In the embodiments of FIGS. 7A-7E and 7H, the surface features are discrete protuberances spaced apart along a row, with a space between adjacent rows. In FIGS. 7F, the surface feature is an angled ramp 75 that extends all the way across the width of the strip. Multiple rows are separated by a space therebetween. FIG. 7G illustrates a similar embodiment to FIG. 7F, except with the angle and spacing of the surface features changed relative to those in FIG. 7G. FIG. 7H is similar to the embodiment of FIG. 7D, except that the cylindrical-like protuberances extend further outward and away from the strip. In any of these embodiments, the surface features are molded, machined or otherwise formed into a strip. The strip may be thin and flexible so that it conforms to the retractor blade surface. Thus, if the retractor blade is curved, the strip will curve with the retractor blade curve. In preferred embodiments, the back of the strip has adhesive for coupling with the retractor blade. The strips may be easily attached and removed as required during a surgical procedure.

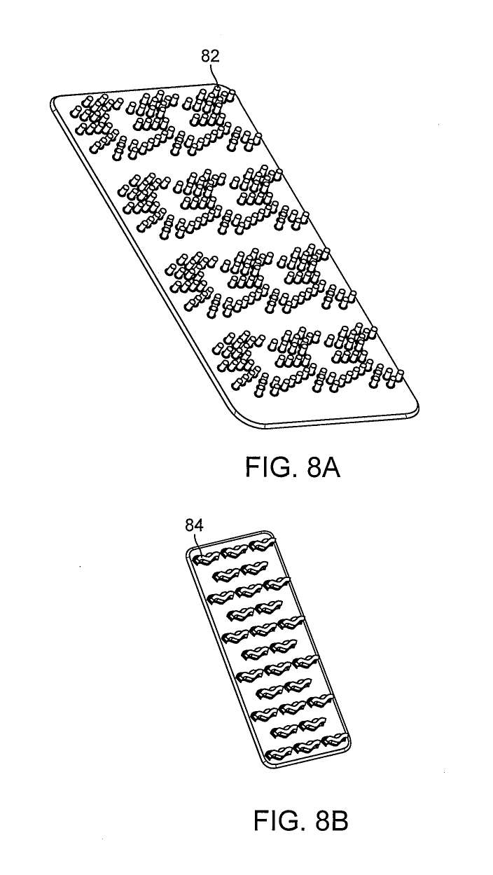

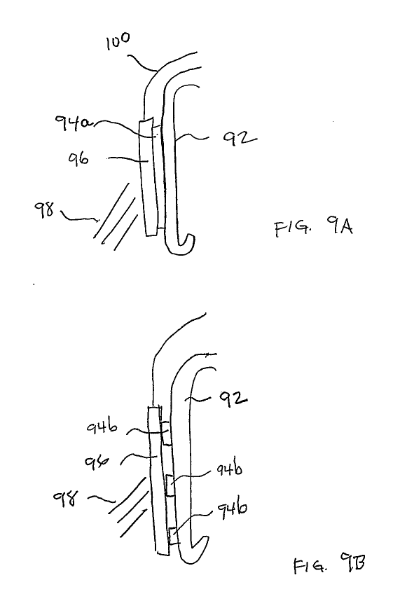

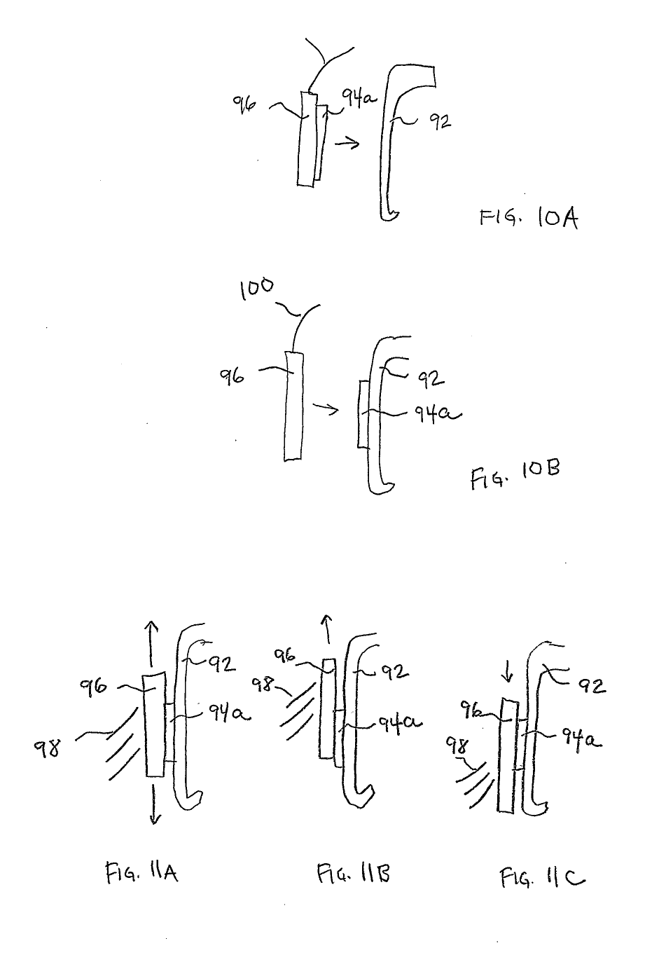

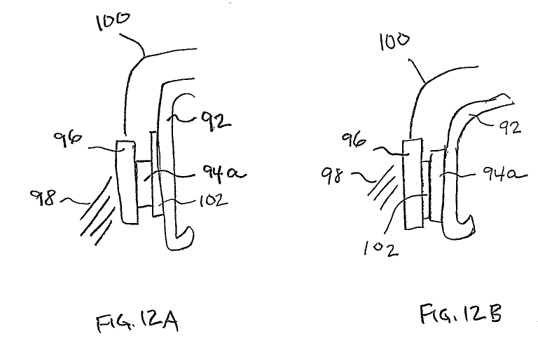

FIGS. 8A-8B illustrate still other embodiments of gripping features which may be included on a strip. In FIG. 8A, a plurality of cylindrical-like protuberances form fan-like patterns on the strip. In FIG. 8B, the protuberances 84 form a pattern having peaks and valleys. One of skill in the art will appreciate that any number of shapes and configurations for the grasping features may be used.

Tissue Illumination.

As previously mentioned, current commercial illuminated retractors do not always include any easy way for easily adjusting the illuminating element. One possible solution involves the use of magnets. The magnets may be integrated into the illumination element or they may be integrated into the retractor. The magnets are of suitable strength so that the illumination element is secured to the retractor and unwanted movement is prevented during the surgical procedure. Additionally, the magnet strength may be selected to allow easy attachment and detachment of the illumination element from the retractor, thereby allowing repositioning. In some circumstances, the retractor may not be magnetic, and thus tape strips of ferrous metal or other magnetic materials may be applied to either or both the illumination element or the retractor so that the magnet will couple the two elements together.

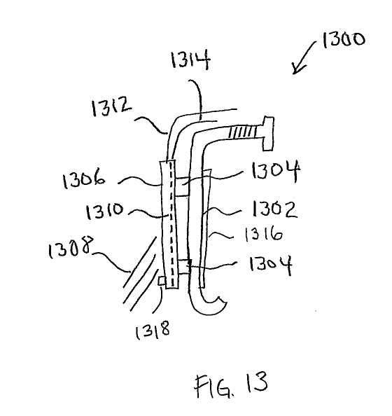

FIG. 9A illustrates one exemplary embodiment where an illumination element 96 such as an optical waveguide is magnetically coupled to retractor blade 92 with a long magnet 94a disposed therebetween. While this embodiment preferably includes an optical waveguide, one of skill in the art will appreciate that any number of illumination elements such as LED lights, incandescent lights, fiber optics, etc. may be used instead of an optical waveguide. A fiber optic or other light input 100 is coupled to the optical waveguide and light 98 is extracted and directed to a target. FIG. 9B illustrates an alternative embodiment where instead of a single continuous magnet extending from a proximal portion of the optical waveguide and retractor blade to a distal portion of the optical waveguide and retractor blade, several smaller, discrete magnets 94b are disposed between the optical waveguide and the retractor blade. This forms spaces between the waveguide and the retractor blade. Thus, in any of these embodiments, the waveguide may be positioned more proximally or more distally relative to the retractor blade, or as previously mentioned, the waveguide (or any other optical illuminator) may be moved laterally or medially along the width of the retractor blade, or the waveguide may be rotated.

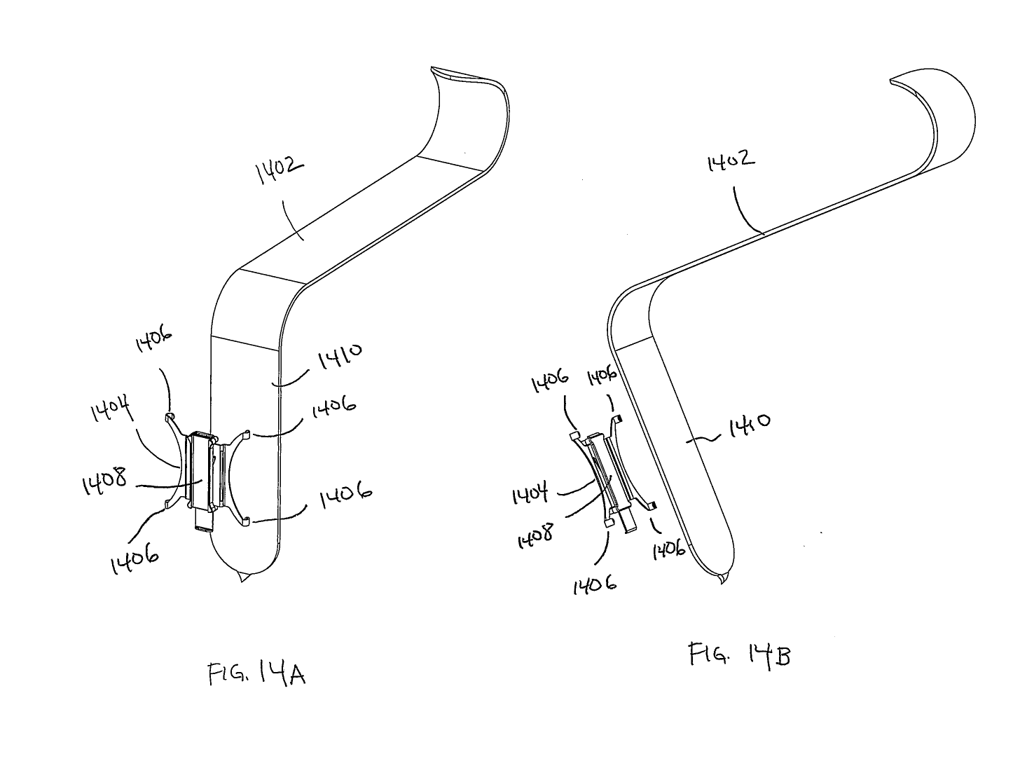

The magnet may be fixedly or releasably coupled to the illumination element or the retractor. For example, in FIG. 10A, the magnet 94a is pre-attached to the illumination element 96, while in FIG. 10B, the magnet is pre-attached to the retractor blade 92. The magnet may be attached using any number of techniques known in the art including adhesives, fasteners, snap fits, press fits, etc. Additionally, if the illumination element or the retractor blade is ferromagnetic, then the magnet may be magnetically coupled thereto.

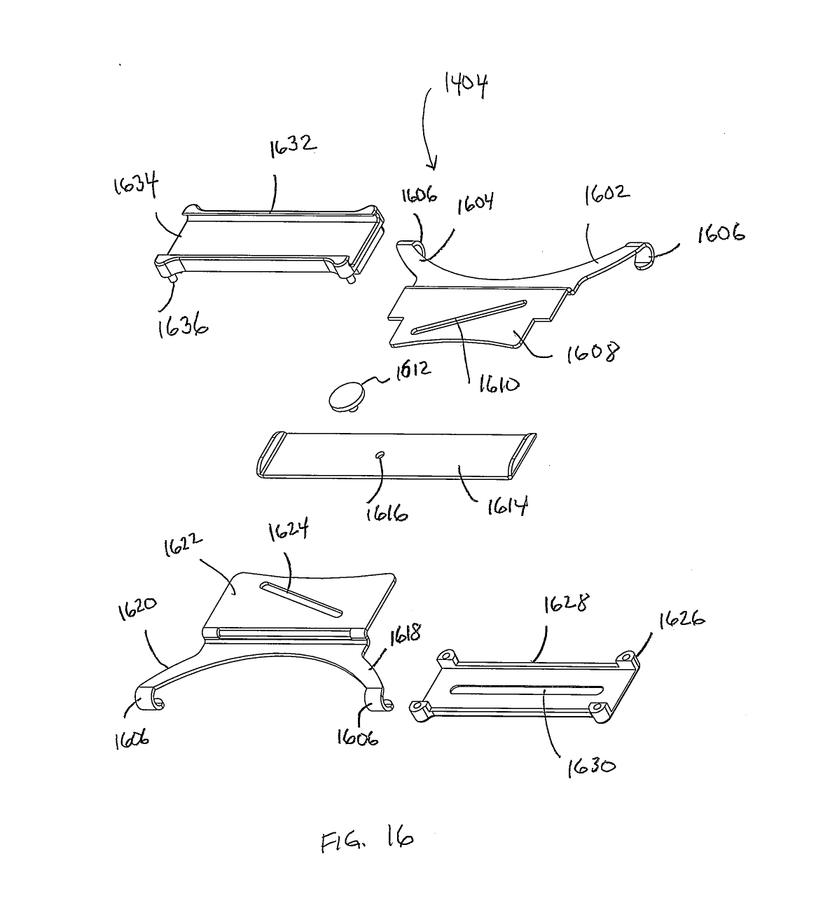

In embodiments where the illumination element or the retractor blade is non-magnetic, a ferromagnetic adhesive strip may be used to help couple the magnet to either element. For example, in FIG. 12A, a ferromagnetic strip 102 is adhesively coupled to the retractor blade 92. Thus, the magnet 94a may be magnetically coupled to the ferromagnetic strip 102, resulting in magnetic coupling between the optical waveguide 96 and the retractor blade 92. FIG. 12B illustrates the opposite configuration where the ferromagnetic strip 102 is adhesively coupled to the waveguide 96 thereby allowing magnetic coupling between the waveguide 96 and the retractor blade 92.

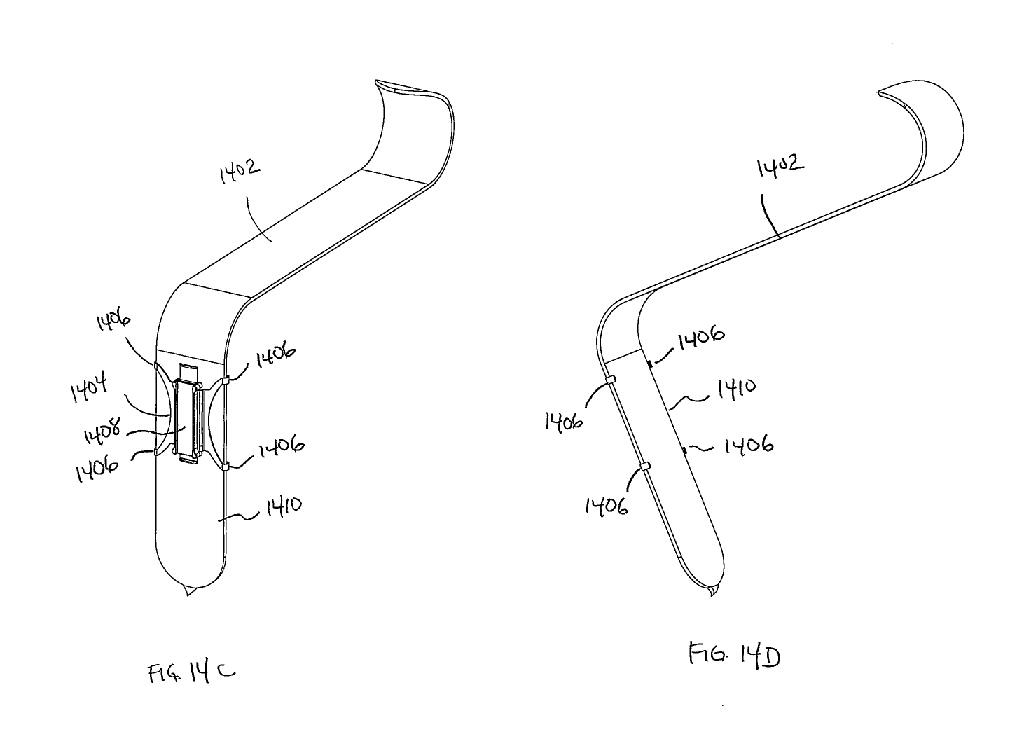

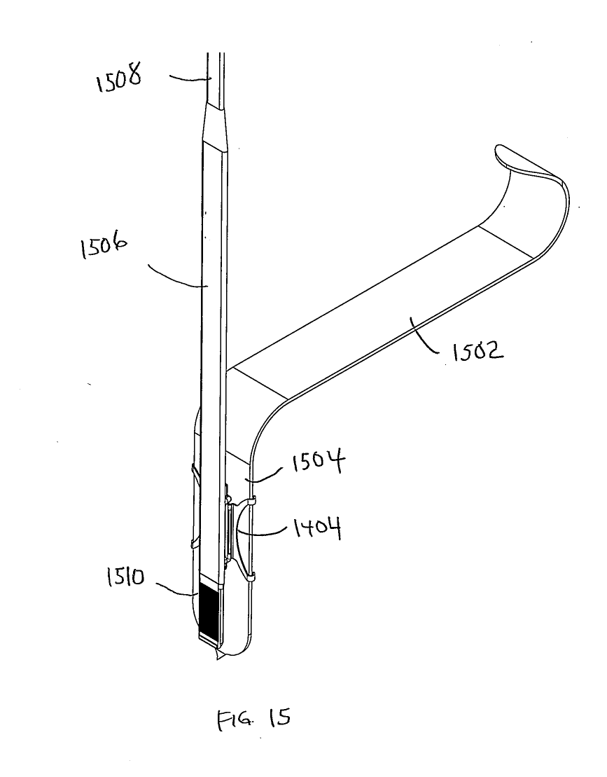

As previously discussed above, once the illumination element such as waveguide 96 is magnetically coupled via magnet 94a to retractor blade 92, as illustrated in FIG. 11A, the illumination element position may be adjusted. For example, in FIG. 11B, the optical waveguide 96 is moved proximally relative to the retractor blade so that the light 98 pattern emitted from the waveguide will be raised, and in FIG. 11C the light 98 pattern is lowered when the waveguide 96 is advanced distally relative to the retractor blade.

The various features disclosed herein may be mixed, matched or substituted with one another. Thus, for example, any of the magnetic coupling embodiments may be combined with any of the tissue gripping features. Additional details on various retractors and optical waveguides are disclosed in U.S. patent application Ser. Nos. 11/654,874; 11/397,446; 11/715,247; 12/188,055; 11/923,483; 12/191,164; 12/616,095; 13/253,785; the entire contents of which are incorporated herein by reference. Any of the features disclosed in this application may be used in conjunction with or substituted with any of the features disclosed in the above referenced patents and applications incorporated by reference.

Smoke Evacuation and Imaging

Any of the embodiments disclosed in this specification may optionally include smoke evacuation features or an imaging element, or both. FIG. 13 illustrates an exemplary device having both features. The illuminated retractor 1300 includes a retractor blade 1302, magnetic coupling elements 1304, an illumination element 1306 such as an optical waveguide for illuminating the surgical field with light 1308 extracted from the illumination element. A smoke evacuation element 1310 is also optionally included. The smoke evacuation element 1310 may be a channel in the illumination element that can be fluidly coupled to an external source of vacuum, or it may be a suction tube which is fixedly or releasably attached to the illumination element, the retractor blade, or any other portion of the device. The smoke evacuation element allows suction to be delivered to the distal portion of the device so that smoke or other noxious fumes may be removed from the surgical field, especially use of an electrosurgical device. Additionally, an optional imaging element 1318 may be fixedly or releasbly coupled to the illumination element, the retractor blade or any other part of the device. Exemplary illumination elements include fiber optics, charge coupled display devices, CMOS devices, camera, as well as other imaging elements known in the art. The imaging element allows an image of the surgical field to be projected to an external display device or electronically stored. Optional gripping elements 1316 such as those described in this specification may also be used in the device.

Retractor Holders

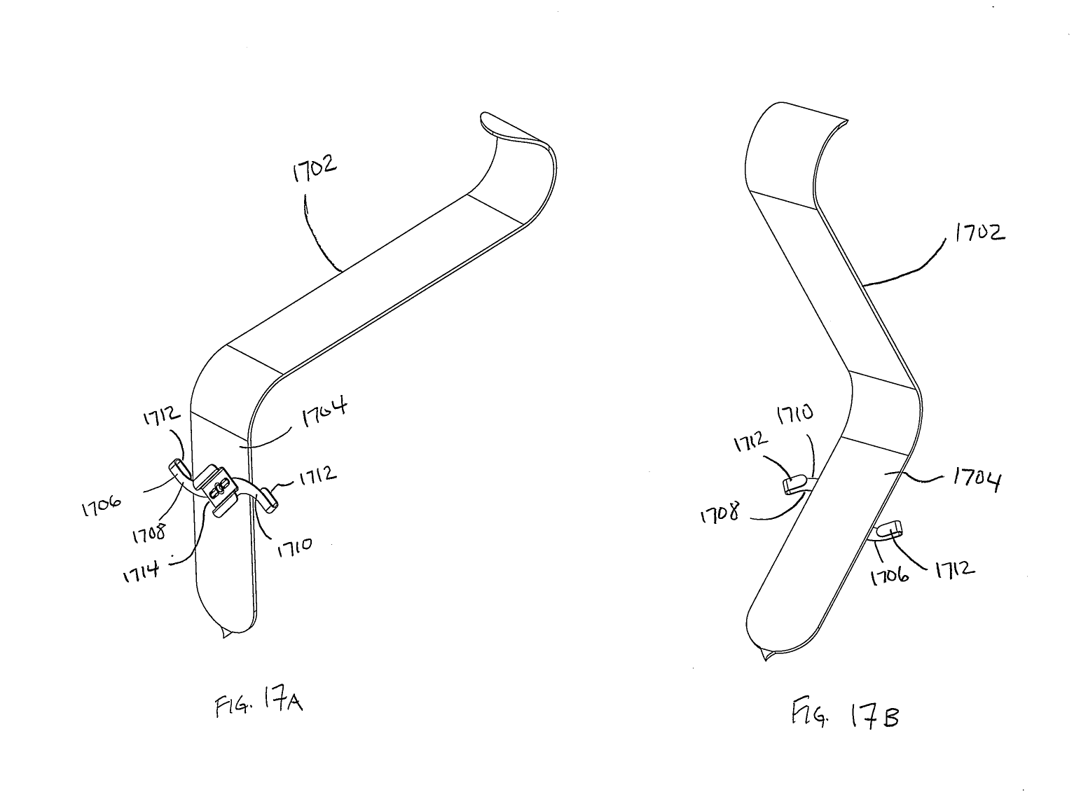

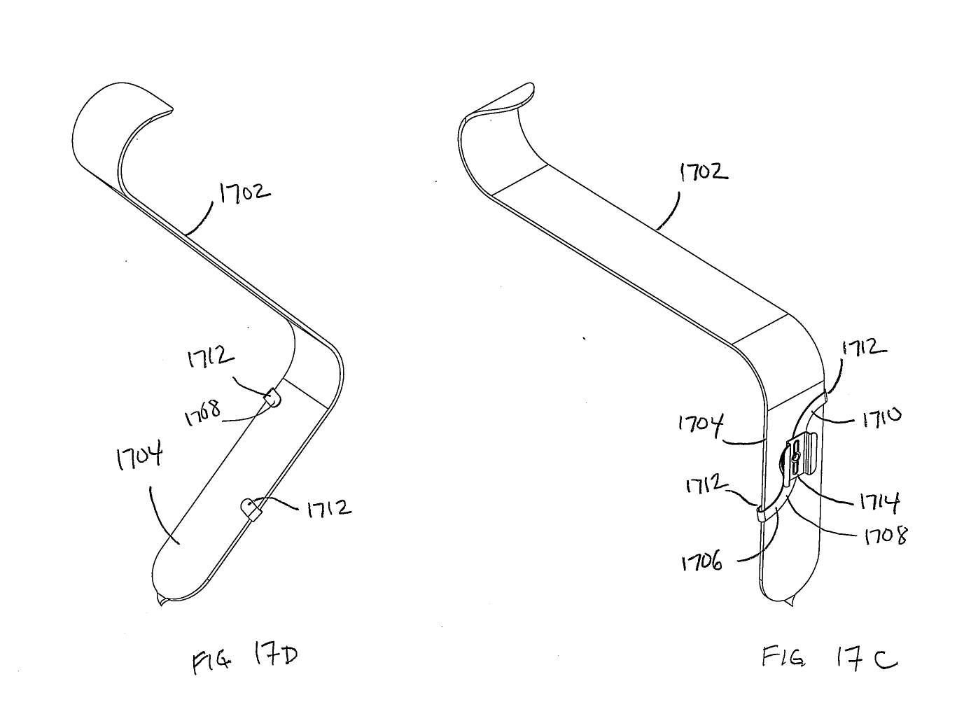

In addition to using magnets to hold a retractor blade, other mechanical coupling devices may be used to hold a retractor blade. For example, FIGS. 14A-14D illustrate an exemplary embodiment of a clip 1404 that may be used to hold a retractor blade 1410 on a surgical retractor 1402. There are many configurations of surgical retractors, therefore it would be desirable to provide a universal adapter that can easily be attached to many different retractor blades. Since most surgical retractors include a flat blade portion, the present embodiment uses this structure for engagement. The clip 1402 may be adjusted so that it can accommodate different sizes of retractor blades.

FIG. 14A illustrates the retractor 1402 having flat retractor blade 1410. The unattached clip 1404 includes four arms 1406 or two opposed pairs of arms with curved tabs on the ends of the arms. The curved tabs are sized so that the tab can fit over the edge of the retractor blade 1410. Another engagement mechanism 1408 on the top surface of the clip 1404 allows another surgical instrument such as an illumination element to easily be attached to the clip and thereby also attached to the retractor blade. FIG. 14B also shows the clip 1404 unattached from the retractor blade 1410 but with the view slightly rotated in the clockwise direction. FIG. 14C shows the clip 1402 attached to the retractor blade 1410, and FIG. 14D illustrates the same configuration from the back side of the retractor blade.

FIG. 15 illustrates a surgical instrument, here an illumination element 1506 coupled to the clip 1404 which is in turn coupled to the retractor blade 1504 on surgical retractor 1502. The illumination element is preferably a non-fiber optic optical waveguide having surface features 1510 adjacent a distal end to extract and direct light from the illumination element to the surgical field. The light input cable 1508 maybe releasably coupled to the proximal portion of the illumination element 1506, or it may be integrally coupled by butt coupling, over molding, potting, etc. In this embodiment the light input is a fiber optic bundle that flattens out near its distal end to form a ribbon like configuration that engages the proximal end of the illumination element. The clip may be adjusted to accommodate for varying widths of the surgical retractor. Additional details of the illumination element may be found in U.S. patent application Ser. No. 14/035,583 (Attorney Docket No. 40556-726.201) filed Sep. 24, 2013; the entire contents of which are incorporated herein by reference.

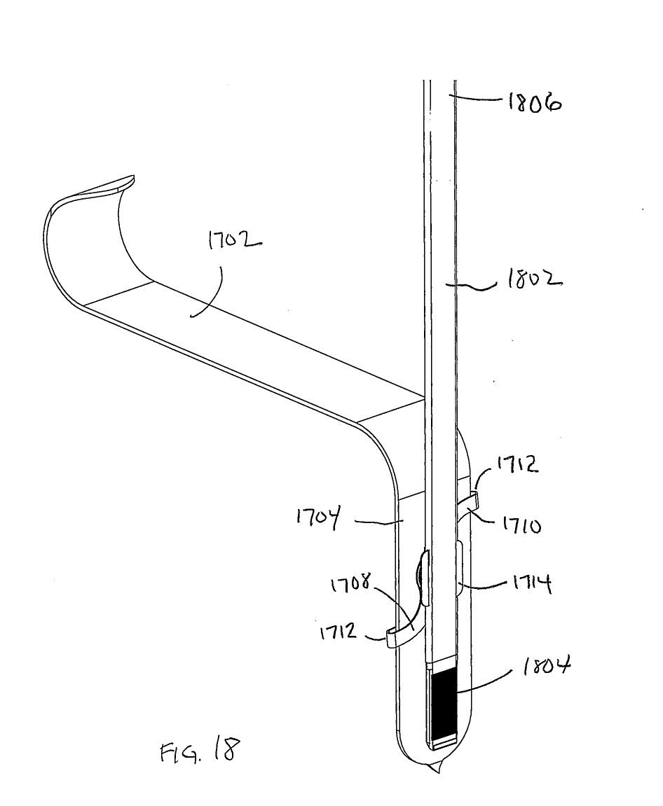

FIG. 16 illustrates an exploded view of the clip 1404 previously described. The clip 1404 includes arms 1602 and 1604 coupled to plate 1608. Curved fingers or tabs 1606 are on the ends of each arm. The tabs are curved to have adequate space to accommodate the thickness of a retractor blade. A diagonal slot 1610 allows adjustment of the clip 1404. A second set of arms 1618, 1620 each having curved fingers or tabs 1606 are coupled to a second plate 1622 having a diagonal slot 1624 which allows adjustment of clip 1404. The tabs are curved to have adequate space to accommodate the thickness of a retractor blade. A center plate 1614 having a center hole 1616 is disposed between plates 1608 and 1622. A pin 1612 having a cylindrical body and cap is disposed through both slits 1610, 1624 and into hole 1616 in middle plate 1614. Plate 1614 may be actuated forward or backward by an operator. Thus, the plates can slide relative to another, thereby increasing or decreasing the width of the clip. For a small width retractor blade, the clip may be collapsed so that the arms fit over the edges of the retractor blade, and similarly for a larger width retractor blade, the clip may be expanded so that that arms fit over the edges of the retractor blade. Upper plate 1632 and lower plate 1628 sandwich all the components therebetween. Feet 1636 form standoffs and are configured to engage the receptacles 1626 on the bottom plate. A linear slot 1630 on the bottom plate also helps the arms slide inward toward, or outward away from one another. A slot 1634 is sized to slidalby receive a surgical instrument such as an illumination element like an optical waveguide. Other instruments may also be coupled to the clip. Thus, clip 1404 is adapted to accommodate a number of different sized retractor blades.

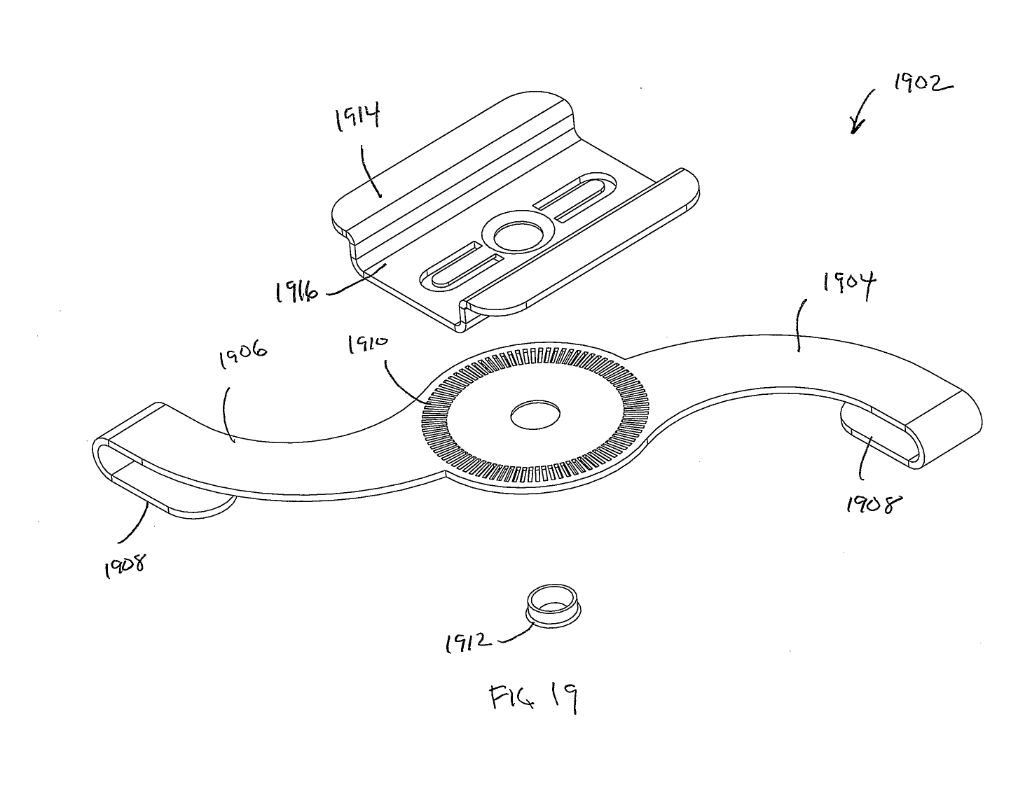

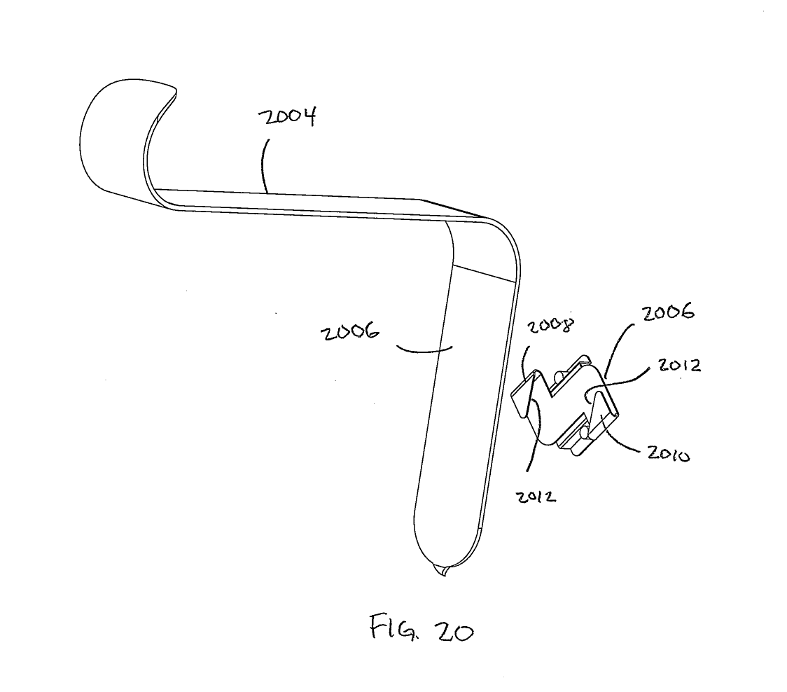

FIGS. 17A-17D illustrate another exemplary embodiment of a clip for engaging and holding a retractor. Clip 1706 includes a first arcuate arm 1708 and a second arcuate arm 1710. Both arms include tabs or fingers 1712 that are bent section having a space wide enough to accept an edge of retractor blade 1704 on retractor 1702. A channel 1714 allows other surgical instruments such as an illumination element like a non-fiber optic optical waveguide to be coupled to the clip 1706. In FIG. 17A, the clip is angled relative to the retractor blade 1704 to allow the blade 1704 to be inserted between the ends of the arms 1708, 1710. FIG. 17B illustrates the back of the retractor blade 1704 when the clip 1706 is disposed over the blade 1706. The clip is then rotated preferably counter clockwise to tighten the clip into engagement with the retractor blade. The tabs capture the retractor blade between the tab and the arm as seen in front view FIG. 17C and rear view FIG. 17D. Preferably the width between arms is sized to match a specific retractor blade, but the clip may be rotated slightly more or slightly less to capture wider or thinner retractor blades.

FIG. 18 illustrates an exemplary embodiment where a surgical instrument is coupled to a retractor blade using a clip. Here, the surgical instrument is a non-fiber optic, optical waveguide 1802 having surface features near the distal end for extracting and directing light from the waveguide toward a surgical field. The proximal portion of the waveguide 1806 is coupled to a light input in substantially the same way previously described with respect to FIG. 15. In this embodiment, or any other embodiment disclosed herein, an imaging element or smoke or fume evacuation element, such as those previously described above may also be included.

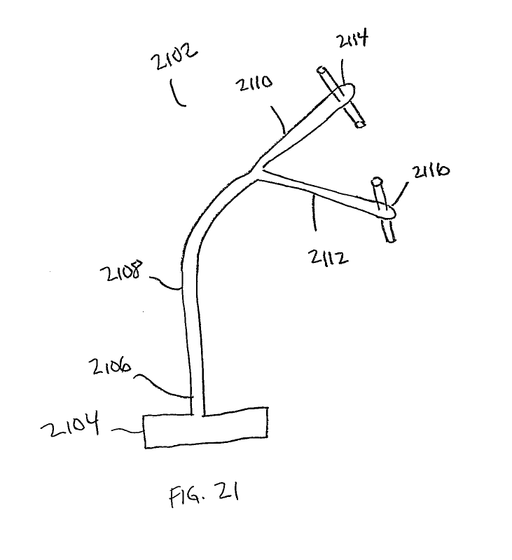

FIG. 19 illustrates an exploded view of a clip 1902 which is generally the same as the clip in FIGS. 17A-17D or FIG. 18. It includes two arcuate arms 1904, 1906 coupled together via a circular plate having a ratchet-like surface and a central hole. The ends of the arms include bent over tabs 1908 with a gap sized to accommodate certain retractor blade thicknesses. An upper plate 1914 is disposed over the circular plate and has a channel 1916 for receiving the surgical instrument such as an optical waveguide. A snap fit, rivet, pin, or other mechanical fastener 1912 secures the circular plate to the upper plate. The upper plate may rotate relative to the circular plate at intervals defined by the pitch of the ratchet surface.

FIG. 20 illustrates an alternative exemplar embodiment of a clip for holding a retractor blade. The clip 2006 includes arms 2008, 2010 opposite one another with tabs or fingers that are bent over sections having an angled planar section 2012 for engaging the blade 2006 of the surgical retractor 2004. Unlike the previous embodiment, the arms are straight linear arms rather than arcuate arms. The clip 2006 is attached to the retractor blade in a similar manner by first angling the clip relative to the retractor blade so that the blade is received between the arms. The clip is then rotated so that the tabs or fingers capture the edges of the retractor blade. A channel (not seen) similar to that previously described above in FIG. 19 is sized to receive a surgical instrument such as an optical waveguide.

Actuatable Arms

Often the retractor blades are held by a physician or assistant during a procedure. However, the retractors may also be fixed to an arm in order to free up the surgeon's hands. Often these arms are fixed to the operating room table or an adjacent surface such as an operating room wall. These arms often require adjustment to reach the retractor and to hold them in the desired location and this can be cumbersome. FIG. 21 illustrates an exemplary embodiment of an actuatable arm 2102 that is advantageous for holding multiple instruments simultaneously. The actuatable arm 2102 includes a base 2104 which can be releasably or fixedly coupled to an operating room table or adjacent surface. A main trunk 2108 is coupled to the base and it may be adjusted using adjustment mechanism 2106. The main trunk 2108 branches off into one, or two, or more arms 2101, 2112 each having a holding element 2114, 2116 for grasping a surgical instrument such as a pin which can be coupled to the clips previously described above thereby allowing a retractor and illumination element to be held by the arm. The main trunk 2108 and/or the arms 2110, 2112 may be actuatable from a flexible configuration to a rigid configuration. In the flexible configuration, the main trunk or the arms may be positioned into a desired shape or location. In the rigid configuration, the main trunk or arms hold their shape rigidly thereby maintaining position of the surgical instrument. Actuation of the main trunk or arms may be accomplished using any number of techniques such as pneumatically, hydraulically, using stiffening wires, locking articulations, motors, etc. Any of the surgical instruments described in this specification may be coupled to such an arm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical retractor for retracting tissue, said retractor comprising:
a retractor blade having a proximal end and a distal end;
a gripping element releasably coupled to the retractor blade, the gripping element having a textured surface adapted to engage and grip the tissue, and wherein the gripping element is adapted to be released from the retractor blade before, during, or after surgery.

2. The retractor of claim 1, wherein the gripping element is adhesively coupled with the retractor blade.

3. The retractor blade of claim 1, wherein the retractor blade has a rear surface for engaging the tissue and a front surface opposite thereto, and wherein the gripping element is disposed on the rear surface.

4. The retractor blade of claim 1, wherein the gripping element is continuously disposed on the retractor blade.

5. The retractor blade of claim 1, wherein the gripping element comprises a plurality of gripping pads disposed in a plurality of discrete regions on the retractor blade.

6. The retractor blade of claim 1, wherein the gripping element extends substantially all the way between the proximal and distal ends of the retractor blade.

7. The retractor blade of claim 1, wherein at least a portion of the retractor blade remains uncovered by the gripping element.

8. The retractor blade of claim 1, wherein the textured surface is uniformly textured along the gripping element.

9. The retractor blade of claim 1, wherein the textured surface comprises a first textured region and a second textured region, and wherein the first textured region is textured differently than the second textured region.

10. The retractor blade of claim 9, wherein the first textured region is closer to the proximal end of the retractor blade than the second texture region, and wherein the second textured region is closer to the distal end than the first textured region, and wherein the first textured region has a finer texture than the second textured region, and wherein the second textured region has a coarser texture than the first textured region.

11. The retractor blade of claim 1, wherein the textured surface is configured to engage and grasp fat, muscle, blood vessels, nerves, or skin.

12. The retractor blade of claim 1, wherein the textured surface comprises a plurality of gripping elements protruding outward from the retractor blade.

13. The retractor blade of claim 12, wherein the plurality of gripping elements extend outward at an angle relative to an outer surface of the retractor blade.

14. The retractor blade of claim 12, wherein the plurality of gripping elements extend outward orthogonally relative to an outer surface of the retractor blade.

15. The retractor blade of claim 12, wherein the plurality of gripping elements is arranged into a plurality of rows disposed on the retractor blade.

16. The retractor blade of claim 12, wherein the plurality of gripping elements is arranged into a symmetric pattern.

17. The retractor blade of claim 1, wherein the gripping element is flexible and configured to conform to the retractor blade when disposed thereon.

18. The retractor blade of claim 1, wherein the retractor blade has a recessed region for receiving the gripping element such that the gripping element is substantially flush with the retractor blade.

19. The retractor blade of claim 1, further comprising an optical waveguide coupled to the retractor blade, the waveguide configured to deliver light toward the tissue.

20. The retractor blade of claim 1, further comprising a handle coupled to the proximal end of the retractor blade.

21. The retractor blade of claim 1, further comprising a smoke evacuation element adjacent a distal end of the retractor blade, the smoke evacuation element configured to remove fumes from a surgical field.

22. The retractor blade of claim 1, further comprising an imaging element adjacent a distal end of the retractor blade, the imaging element configured to provide an image of a surgical field.

23. A surgical method for retracting tissue, said method comprising:
providing a surgical retractor blade having a proximal end and a distal;
selecting a gripping element from a plurality of different gripping elements;
releasably coupling the selected gripping element with the surgical retractor blade;
advancing the surgical retractor blade toward the tissue;
engaging the gripping element with the tissue; and
retracting the surgical retractor blade thereby retracting the tissue.

24. The method of claim 23, wherein releasably coupling comprises adhesively coupling the selected gripping element with the surgical retractor blade.

25. The method of claim 23, further comprising uncoupling the selected gripping element from the surgical retractor blade.

26. The method of claim 25, further comprising:
selecting a second gripping element from the plurality of gripping elements; and
releasably coupling the second selected gripping element with the surgical retractor blade.

27. The method of claim 23, wherein retracting the tissue comprises retracting fat, muscle, blood vessels, nerves, or skin.

28. The method of claim 23, wherein the surgical retractor blade has an optical element coupled thereto, the method further comprising illuminating the tissue with light from the optical element.

29. The method of claim 28, wherein the optical element comprises an optical waveguide.

30. The method of claim 23, wherein releasably coupling the selected gripping element comprises disposing the selected gripping element substantially all the way between the proximal and distal ends of the surgical retractor blade.

31. The method of claim 23, wherein releasably coupling the selected gripping element comprises maintaining a portion of the surgical retractor blade uncovered by the selected gripping element.

32. The method of claim 23, wherein the selected gripping element has a first textured region and a second textured region, the first textured region having a texture different than the second textured region, and wherein the releasably coupling the selected gripping element comprises disposing the first textured region at a first end of the surgical retractor blade and disposing the second textured region at a second end of the surgical retractor blade opposite the first end.

33. The method of claim 32, wherein the first textured region has a finer texture than the second textured region, and wherein the second textured region has a coarser texture than the first textured region, and wherein releasably coupling comprises disposing the first textured region adjacent the proximal end of the surgical retractor blade and disposing the second textured region adjacent the distal end of the surgical retractor blade.

34. The method of claim 23, wherein the gripping element comprises a plurality of gripping pads, and wherein releasably coupling comprises affixing the plurality of gripping pads to the surgical retractor blade.

35. The method of claim 23, wherein releasably coupling the selected gripping element with the surgical retractor blade comprises conforming the selected gripping element to the surgical retractor blade.

36. The method of claim 23, wherein releasably coupling comprises disposing the selected gripping element in a recessed region of the surgical retractor blade so that the selected gripping element is substantially flush with the retractor blade.

37. The method of claim 23, further comprising evacuating smoke from a surgical field with a smoke evacuation element disposed adjacent a distal end of the retractor blade.

38. The method of claim 23, further comprising imaging a surgical field with an imaging element disposed adjacent a distal end of the retractor blade.

39. An illuminated surgical retractor for retracting tissue, said surgical retractor comprising:
a retractor blade having a proximal end and a distal end;
an optical illumination element configured to deliver light from the optical illumination element to the tissue; and
a magnetic element coupled to the retractor blade and the optical illumination element so that the optical illumination element is releasably coupled to the retractor blade.

40. The illuminated surgical retractor of claim 39, wherein the magnetic element comprises a plurality of magnets.

41. The illuminated surgical retractor of claim 39, wherein the magnetic element is fixedly attached to one of the optical illumination element or the surgical retractor blade.

42. The illuminated surgical retractor of claim 39, wherein the optical illumination element is adjustably coupled with the surgical retractor blade such that the optical waveguide may be positioned between the proximal and distal ends of the surgical retractor blade.

43. The illuminated surgical retractor of claim 39, wherein the illumination element comprises an optical waveguide.

44. The illuminated surgical retractor of claim 39, further comprising a magnetic adhesive strip disposed between the optical illumination element and the magnetic element, or between the surgical retractor blade and the magnetic element.

45. The retractor blade of claim 39, further comprising a smoke evacuation element adjacent a distal end of the retractor blade, the smoke evacuation element configured to remove fumes from a surgical field.

46. The retractor blade of claim 39, further comprising an imaging element adjacent a distal end of the retractor blade, the imaging element configured to provide an image of a surgical field.

47. A surgical method for retracting tissue in a patient, said method comprising:
providing a surgical retractor blade having a proximal end and a distal end;
providing an optical illumination element;
magnetically coupling the optical illumination element with the retractor blade;
advancing the retractor blade to the tissue;
retracting the tissue with the retractor blade; and
illuminating the tissue with light from the optical illumination element.

48. The method of claim 47, further comprising adjusting position of the optical illumination element relative to the surgical retractor blade.

49. The method of claim 48, wherein adjusting position comprises:
uncoupling the optical illumination element from the surgical retractor blade;
moving the optical illumination element relative to the surgical retractor blade; and
re-coupling the optical illumination element magnetically with the surgical retractor blade.

50. The method of claim 48, wherein adjusting position comprises moving the optical illumination element between the proximal and distal ends of the surgical retractor blade.

51. The method of claim 48, wherein adjusting position comprises moving the optical illumination element laterally or medially along the surgical retractor blade.

52. The method of claim 48, wherein adjusting position comprises rotating the optical illumination element relative to the surgical retractor blade.

53. The method of claim 47, wherein magnetically coupling comprises coupling the optical illumination element with the surgical retractor blade using a plurality of magnets.

54. The method of claim 47, further comprising affixing a magnetic adhesive strip to the optical illumination elementor the surgical retractor blade.

55. The method of claim 47, further comprising evacuation smoke from a surgical field with a smoke evacuation element disposed adjacent a distal end of the retractor blade.

56. The method of claim 47, further comprising imaging a surgical field with an imaging element disposed adjacent a distal end of the retractor blade.

57. A system for holding a surgical retractor, said system comprising:
a clip comprising a first arm and a second arm opposite the first arm, the first arm having a first engagement element and the second arm having a second engagement element,
wherein the first and second arms are spaced apart by a distance, the distance sized to match a width of a retractor blade, and
wherein the first engagement element is configured to engage a first edge of a retractor blade and the second engagement element is configured to engage a second edge of the retractor blade, thereby attaching the clip to the retractor blade; and
a holding mechanism for holding an illumination element, the holding mechanism coupled to the clip.

58. The system of claim 57, further comprising the retractor blade.

59. The system of claim 57, further comprising the illumination element.

60. The system of claim 59, wherein the illumination element comprises a non-fiber optic optical waveguide.

61. The system of claim 57, further comprising:
a third arm and a fourth arm opposite the third arm, the third arm having a third engagement element and the fourth arm having a fourth engagement element,
wherein the third and fourth arms are spaced apart by a distance, the distance sized to match the width of the retractor blade, and
wherein the third engagement element is configured to engage the first edge of the retractor blade and the fourth engagement element is configured to engage the second edge of the retractor blade, thereby attaching the clip to the retractor blade.

62. The system of claim 57, wherein the first arm and the second arm are movable relative to one another thereby adjusting the distance.

63. The system of claim 57, wherein the first arm or the second arm is substantially linear.

64. The system of claim 57, wherein the first arm or the second arm is arcuate.

65. The system of claim 57, wherein the first arm is rotatably engageable with the first edge or the second arm is rotatably engageable with the second edge.

66. The system of claim 57, wherein the first engagement element or the second engagement element comprise tabs.

67. The system of claim 57, wherein the first edge of the retractor blade is on an opposite side of the retractor blade second edge.

68. The system of claim 57, further comprising an arm coupleable with the clip, the arm configured to be coupled with an adjacent operating room table surface or other adjacent surface, and wherein the arm is configured to steadily hold the clip and retractor blade coupled thereto.

69. The system of claim 68, wherein the arm is actuatable between a flexible movable configuration and a substantially inflexible immovable configuration for steadily holding the clip and retractor blade.

70. The system of claim 57, further comprising an imaging element adjacent a distal portion of the retractor blade.

71. The system of claim 57, further comprising a smoke evacuation element for removing smoke or noxious fumes from a surgical site in which the retractor blade is disposed.

72. A method for holding a surgical retractor blade, said method comprising:
providing a surgical retractor blade having a first edge and a second edge opposite the first edge;
providing a clip having a first arm and a second arm opposite the first arm;
engaging the first arm with the first edge of the retractor blade;
engaging the second arm with the second edge of the retractor blade, thereby holding the surgical retractor blade with the clip; and
attaching a surgical instrument to the clip.

73. The method of claim 72, wherein engaging the first arm with the first edge comprises engaging a tab on the first arm with the first edge.

74. The method of claim 72, wherein engaging the second arm with the second edge comprises engaging a tab on the second arm with the second edge.

75. The method of claim 72, wherein attaching the surgical instrument comprises attaching an illumination element to the clip.

76. The method of claim 75, further comprising illuminating a surgical field with light from the illumination element.

77. The method of claim 72, further comprising imaging a surgical field with an imaging element adjacent a distal end of the retractor blade.

78. The method of claim 72, further comprising evacuating smoke or fumes from a surgical field with a smoke evacuation element adjacent a distal end of the retractor blade.

79. The method of claim 72, further comprising coupling the clip to an arm, the arm coupled to an operating room table or adjacent surface, the arm holding the clip and surgical instrument in a substantially fixed position.

80. The method of claim 72, wherein engaging the first arm or engaging the second arm comprises rotating the first arm into engagement with the first edge or rotating the second arm into engagement with the second edge.
